(19) United States
(12) Patent Application Publication
(10) Pub. No.: US 2026/0334887 A1
(43) Pub. Date: Oct. 29, 2026

(54) SYSTEM FOR MONITORING AND PROVIDING ALERTS OF A FALL RISK BY PREDICTING RISK OF EXPERIENCING SYMPTOMS RELATED TO ABNORMAL BLOOD PRESSURE(S) AND/OR HEART RATE

(71) Applicant: UNIVERSITY OF PITTSBURGH-OF THE COMMONWEALTH SYSTEM OF HIGHER EDUCATION, PITTSBURGH, PA (US)

(72) Inventor: EUNICE EUN YOUNG YANG, PITTSBURGH, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 19/632,878

(22) Filed: Mar. 30, 2026

Related U.S. Application Data
(63) Continuation-in-part of application No. 18/762,823, filed on Jul. 3, 2024, now abandoned.
Application No. 18/762,823 is a division of application No. 17/053,425, filed on May 7, 2019, now abandoned.
(60) Provisional application No. 62/668,428, filed on May 8, 2018.

(51) Int. Cl. A61B 5/00; A61B 5/021; A61B 5/024; A61B 5/11; A61B 5/318

(52) U.S. Cl. CPC A61B 5/7275; A61B 5/021; A61B 5/02405; A61B 5/1117; A61B 5/318; A61B 5/6801; A61B 5/7264; A61B 5/7405; A61B 5/742; A61B 5/7455

(57) ABSTRACT

A method of predicting the risk of experiencing symptoms related to abnormal blood pressure and/or heart rate that includes obtaining subject heart rate variability data representing a number of HRV parameters, wherein the subject HRV data is generated based on heartbeat data obtained from an individual wearing a heart parameter sensor, providing the subject HRV data as an input to an artificial intelligence system, wherein the artificial intelligence system has been previously trained using training and test HRV data representing the number of HRV parameters obtained from a plurality of test subjects, and analyzing temporal data changes in or indicated by the subject HRV data in the artificial intelligence system to determine whether the individual is at risk of experiencing symptoms related to abnormal blood pressure and/or heart rate placing them at risk of a fall.

### CROSS-REFERENCE TO RELATED APPLICATIONS

This is a Continuation application of U.S. Divisional patent application Ser. No. 18/762,823, filed on Jul. 3, 2024, titled “SYSTEM FOR MONITORING AND PROVIDING ALERTS OF A FALL RISK BY PREDICTING RISK OF EXPERIENCING SYMPTOMS RELATED TO ABNORMAL BLOOD PRESSURE(S) AND/OR HEART RATE”, which claims priority from U.S. patent application Ser. No. 17/053,425, filed on Nov. 6, 2020, titled “SYSTEM FOR MONITORING AND PROVIDING ALERTS OF A FALL RISK BY PREDICTING RISK OF EXPERIENCING SYMPTOMS RELATED TO ABNORMAL BLOOD PRESSURE(S) AND/OR HEART RATE” (abandoned), which was a U.S. National Stage Application under 35 U.S.C. § 371 of International Application No. PCT/US2019/031042, filed on May 7, 2019, titled “SYSTEM FOR MONITORING AND PROVIDING ALERTS OF A FALL RISK BY PREDICTING RISK OF EXPERIENCING SYMPTOMS RELATED TO ABNORMAL BLOOD PRESSURE(S) AND/OR HEART RATE”, which claims priority under 35 U.S.C. § 119(e) from U.S. Provisional Patent Application No. 62/668,428, filed on May 8, 2018, titled “ORTHOSTATIC HYPOTENSION ALERT SYSTEM”, the contents of which are incorporated herein by reference.

### BACKGROUND OF THE INVENTION

### 1. Field of the Invention

The disclosed concept relates to medical alert systems, and, in particular, to a system and method for providing an alert of a fall risk. More particularly, in certain embodiments, the disclosed concept relates to a system and method for detecting abnormal blood pressure drops, and/or increase or decrease in pulse rate, in real-time (as described herein) using individualized criteria and providing actionable cues to mitigate a potential injurious fall.

### 2. Description of the Related Art

According to the World Health Organization, falls are the second leading cause of accidental or unintentional injury deaths worldwide. It is estimated that 205 million falls occurred in 2015, with 37.3 million falls resulting in the need for medical attention and 646,000 falls resulting in death. Over 80% of the deaths are from low- and middle-income countries.

There are multiple reasons and causes for these falls. The disclosed concept addresses falls caused by abnormal blood supply to brain resulting in symptoms such as dizziness or even fainting. To illustrate the significance of this problem, fall statistics only due to orthostatic hypotension or OH (i.e. dizziness upon standing) is discussed. It is estimated that ⅓ of all adults 65 years of age and older experience OH, which places them at a risk for falling. Approximately 5% of people between the ages of 45 and 65 living in the community experience falls due to OH. In professional care settings (e.g., in hospitals, skilled nursing centers, assisted living environments, or other care facilities), the fall rate due to OH is as high as 64%.

To minimize injuries after a fall, attempts to provide prompt medical care is facilitated by the use of fall prevention devices such as bed, chair, and floor mat alarms that detect fall impacts. High fall risk individuals are frequently monitored by facility staff or a sitter who remains in the same room to supervise the individual. There are even solutions where a person at an off-site location monitors older adults via a PC screen and manually places calls to facilities notifying them of a high fall-risk situation (i.e. someone has made a motion to leave their bed). With the number of older adults expected to more than double by 2050 and to more than triple by 2100, rising from 962 million globally in 2017 to 2.1 billion in 2050 and 3.1 billion in 2100, these solutions are not adequate and not scalable.

Abnormal blood pressure drops and/or heart rate results in decreased blood flow to the brain resulting in individuals to experience fall risk symptoms such as light-headedness, visual blurring, dizziness, generalized weakness, fatigue, cognitive slowing, leg buckling, coat-hanger ache, and gradual or sudden loss of consciousness. When an individual assumes an upright position from a lying or sitting position, approximately 500 to 1,000 ml of blood pools to the legs and internal abdominal organs. Similar pooling occurs when an individual stands for a long period of time. In healthy individuals, the physiological response consists of an increase in heart rate and blood pressure so that the blood pooled in the lower extremities can be redistributed to vital organs including the brain, thereby avoiding fall risk symptoms. However, due to diseases and natural effects of aging, the physiological response of older adults can be delayed, inadequate, or non-existent, placing them at a high risk of an injurious fall.

Abnormal blood pressures and heart rate upon assuming an upright position or while in an upright position is defined by a progressive and sustained fall in systolic BP from baseline value ≥20 mmHg and/or diastolic BP ≥10 mmHg, or an increase in heart rate of ≥30 beats per minute within three minutes of standing. This is illustrated in FIG. 1 for an individual initially in supine and then standing up. While in a supine position, the individual exhibits an average of 150 mmHg systolic blood pressure. At 960 seconds, the individual transitions to a standing position and blood pressures begin to drop quickly due to blood pooling to the lower extremities. Approximately 7 seconds later, the individual completes his/her transition to an upright position and experiences a systolic blood pressure drop of approximately 70 mmHg. The individual's automatic physiological response causes the heart rate to increase, resulting in a quick increase in both the systolic and diastolic blood pressures. What is problematic is that at 980 seconds, the blood pressure remains more than 20 mmHg and 10 mmHg lower in systolic and diastolic pressures, respectively, indicating that s/he is in an OH state and will most likely be experiencing fall risk symptoms. Even more troubling is that even after 2.5 minutes (1110 seconds), the blood pressures remain low, placing the individual at a continued risk of experiencing a fall.

Clinically, the inability for the body to compensate for orthostatic stress by regulating blood pressure and/or heart rate is a sign of poor baroreflex sensitivity and that the autonomic nervous system (ANS) of the individual is not working well. To elaborate, the ANS is located in the brain and it auto-regulates organs and nerves to keep an individual's body in continuous healthy balance, also known as homeostasis. ANS consists of two branches called the parasympathetic nervous system (PNS) and sympathetic nervous system (SNS). In healthy individuals at rest, both the SNS and PNS are active, with SNS being the dominant system. ANS dysfunction can be caused by or is a result of factors such as, but not limited to, natural age-related physiological decline, hypertension medication called vasodilators, decrease in blood volume due to dehydration, Parkinson's disease, and damaging effects on the autonomic system due to high levels of glucose in people with diabetes. Furthermore, individuals with impaired cardiac function due to structural heart disease, impaired cardiovascular adrenergic function (neurogenic OH), pure autonomic failure (PAF), multiple system atrophy (MSA), alcohol consumption, and exposure to heat have ANS dysfunction placing them at higher risk of experiencing fall risk symptoms.

Clinically, various diseases related to ANS dysfunction are diagnosed through the use of a tilt table and a procedure called the head-up tilt (HUT) test or a tilt table test (TTT). In such test, a person in supine position is strapped to a table and kept at rest for ≥5 minutes. Blood pressure and heart rate are measured and are used as baseline values. The individual is then rotated 60-70 degrees and kept passive in this position for 20 minutes to 40 minutes maximum. During this passive position, indications of abnormal blood pressures and/or heart rates signify various diseases related to ANS dysfunctions such as but not limited to OH, syncope, and postural orthostatic hypotension. The reported sensitivity, specificity, and reproducibility of such testing ranges greatly (e.g., from as low as 55% to as high as 96%). This large range is attributed to the fact that symptoms are transient, and that often results in individuals being misdiagnosed. When a person is under professional care and is susceptible to ANS dysfunction, s/he is placed under greater observation and told to be careful. When individuals are misdiagnosed or undiagnosed, this can compromise how they are treated medically as well as how they are supervised when they are admitted to facilities such as hospitals and various forms of long-term care. There is no current commercially available system to predict when someone would experience fall risk symptoms associated with ANS dysfunction (i.e. abnormal blood pressure and/or heart rate), thus driving the motivation for the disclosed concept described below.

### SUMMARY OF THE INVENTION

In one embodiment, a method of predicting the risk of experiencing symptoms related to abnormal blood pressure and/or heart rate is provided. The method includes obtaining subject heart rate variability (HRV) data representing a number of HRV parameters, wherein the subject HRV data is generated based on heartbeat data obtained from an individual wearing a heart parameter sensor. The method further includes providing the subject HRV data as an input to an artificial intelligence system, wherein the artificial intelligence system has been previously trained using training and test HRV data representing the number of HRV parameters obtained from a plurality of test subjects. Finally, the method includes analyzing temporal data changes in or indicated by the subject HRV data in the artificial intelligence system to determine whether the individual is at risk of experiencing symptoms related to abnormal blood pressure and/or heart rate placing them at risk of a fall.

In another embodiment, an apparatus for predicting the risk of experiencing symptoms related to abnormal blood pressure and/or heart rate is provided. The apparatus includes a computer system comprising a number of controllers implementing an artificial intelligence system that has been previously trained using training and test heart rate variability (HRV) data representing a number of HRV parameters obtained from a plurality of test subjects. The artificial intelligence system is structured and configured to obtain subject HRV data representing the number of HRV parameters, wherein the subject HRV data is generated based on heartbeat data obtained from an individual wearing a heart parameter sensor, provide the subject HRV data as an input to the artificial intelligence system, and analyze temporal data changes in or indicated by the HRV data in the artificial intelligence system to determine whether the individual is at risk of experiencing symptoms related to abnormal blood pressure and/or heart rate.

In yet another embodiment, a method of training an artificial intelligence system to predict the risk of experiencing symptoms related to abnormal blood pressure and/or heart rate is provided. The method includes obtaining heartbeat data and blood pressure data from a plurality of test subjects, generating training and test heart rate variability (HRV) data representing a number of HRV parameters from the heartbeat data and the blood pressure data, and training the artificial intelligence system to predict risk of experiencing symptoms related to abnormal blood pressure and/or heart rate using the training and test HRV data based on temporal data changes in or indicated by the training and test HRV data.

In still another embodiment, a system for predicting the risk of experiencing symptoms related to abnormal blood pressure and/or heart rate is provided. The system includes a wearable biometric sensor including a heart parameter sensor structured and configured to generate heartbeat data from an individual wearing the wearable biometric sensor, and a computer system comprising a number of controllers implementing a predictive artificial intelligence system comprising an artificial intelligence system that has been previously trained using training and test heart rate variability (HRV) data representing a number of HRV parameters obtained from a plurality of test subjects. The predictive artificial intelligence system is structured and configured to obtain subject HRV data representing the number of HRV parameters, wherein the subject HRV data is generated based the heartbeat data, provide the subject HRV data as an input to the artificial intelligence system, and analyze temporal data changes in or indicated by the HRV data in the artificial intelligence system to determine whether the individual is at risk of experiencing symptoms related to abnormal blood pressure and/or heart rate.

In another embodiment, a method of outputting a warning signal when a subject is at risk of experiencing symptoms related to abnormal blood pressure and/or heart rate is provided. The method receiving heart rate variability data of a subject wherein the HRV data represents at least one HRV parameter, wherein the person's HRV data is generated based on heartbeat data obtained from a heart parameter sensor worn by the person, and receiving motion data of the person from a motion sensor worn by the person, the motion data being sufficient to distinguish between resting data and vertical rising data, the resting data representing when the subject is resting in the lying or sitting position and the vertical rising data representing when the subject is moving from a lying position to a sitting or standing position or from a sitting position to a standing position, the resting data and the vertical rising data being synced to the HRV data to identify a portion of the HRV data representative of when the person is resting and a portion of the HRV data representative of when the person is vertically rising. The method further includes determining from temporal data changes in or indicated by the subject HRV data associated only with the portion of the HRV data representative of when the person is resting and optionally, the portion of the HRV data representative of when the person is vertically rising whether the person is at risk of experiencing symptoms related to abnormal blood pressure and/or heart rate, and outputting the warning signal when the determining step determines the person is at risk of experiencing symptoms related to abnormal blood pressure and/or heart rate.

In still another embodiment, an apparatus worn by a subject in a resting position to provide a warning signal when the subject is at risk of experiencing symptoms related to abnormal blood pressure and/or heart rate is provided. The apparatus includes a heart rate sensor capable of measuring an interbeat interval at least 100 hertz, the heart rate sensor being attachable to a body of the subject, and a computer in communication with the heart rate sensor. The computer is operative to analyze the data of the heart rate sensor to output the warning signal when the subject is at risk of experiencing symptoms related to abnormal blood pressure and/or heart rate based solely only on the data from the heart rate sensor relating to when the subject is in the resting position.

In yet another embodiment, an apparatus worn by a subject to provide a warning signal when the subject is at risk of falling is provided. The apparatus includes a heart rate sensor capable of measuring an interbeat interval of at least 100 hertz, the heart rate sensor being attachable to a body of the subject, a motion sensor capable of sensing motion data for distinguishing when the subject is resting and when the subject is vertically rising from a lying position to a sitting or standing position or from the sitting position to the standing position, and a computer in communication with the heart rate sensor and the motion sensor and operative to analyze and output the warning signal as to when the subject is at risk of falling based at a minimum, on the data from the heart rate sensor and the motion sensor relating to when the subject is resting and optionally when the subject is rising from the lying position to the sitting or standing position or from the sitting position to the standing position.

In a further embodiment, a method of outputting a warning signal when a subject is at risk of experiencing symptoms related to abnormal blood pressure and/or heart rate is provided. The method includes the steps of receiving heart rate variability (HRV) data of a subject wherein the HRV data represents at least one HRV parameter, wherein the subject's HRV data is generated based on heartbeat data obtained from a heart parameter sensor worn by the subject, determining from temporal data changes in or indicated by the subject HRV data associated only with a portion of the HRV data representative of when the subject is resting and optionally, with a portion of the HRV data representative of when the subject is vertically rising whether the subject is at risk of experiencing symptoms related to abnormal blood pressure and/or heart rate, and outputting the warning signal when the determining step determines the subject is at risk of experiencing symptoms related to abnormal blood pressure and/or heart rate.

In a still further embodiment, a pedal pulse sensor device structured to wrap around a foot of a wearer is provided. The pedal pulse sensor device includes a first laterally extending portion, a second portion that extends transversely from the first portion, wherein at least one of the first portion and the second portion includes a cavity structured to be located along the dorsalis pedis or the posterior tibial arteries of the wearer responsive to the pedal pulse sensor device being wrapped around the foot of the wearer, and a biometric sensor unit held within the cavity, wherein the biometric sensor unit includes a heart parameter sensor.

### BRIEF DESCRIPTION OF THE DRAWINGS

FIG. 1 illustrates the conditions defining abnormal blood pressure response;

FIG. 2 is an exemplary ECG wave signal;

FIG. 3 is an exemplary QRS waveform showing low HRV;

FIG. 4 is an exemplary QRS waveform showing high HRV;

FIG. 5 illustrates the direct correlation between ECG and arterial blood pressure waveforms;

FIG. 6 shows an exemplary 3D frequency spectrogram;

FIGS. 7A and 7B show exemplary PSD graphs from an individual according to an aspect of the disclosed concept;

FIG. 8 is an exemplary Poincaré plot of the dotted window of FIG. 9 according to an aspect of the disclosed concept;

FIG. 9 is a plot of RR vs. time while an individual is supine, in transition from lying to sitting, and then standing during an exemplary test period according to an aspect of the disclosed concept;

FIG. 10 is a 3D frequency spectrogram for the individual during the test of FIG. 9;

FIG. 11 shows power spectral density vs. frequency for a 5-minute window identified as SAMPLE 1 during the test of FIG. 9;

FIG. 12 is a plot of RR vs. time while an individual is supine, in transition from lying to sitting, and then standing during another exemplary test period according to an aspect of the disclosed concept;

FIG. 13 is a 3D frequency spectrogram for the individual during the test of FIG. 12;

FIG. 14 shows PSD vs. frequency for a 5-minute window identified as SAMPLE 1 during the test of FIG. 12;

FIG. 15 is a flowchart that illustrates a method of training and testing an artificial intelligence system (e.g., a machine learning system) according to an exemplary embodiment of the disclosed concept;

FIGS. 16A and 16B show typical ECG waveform and continuous blood pressure signals, respectively, that may be collected during training and of the artificial intelligence system according to the disclosed concept;

FIG. 17 is a schematic diagram of a system for monitoring and providing alerts of a fall risk by predicting risk of experiencing symptoms related to abnormal blood pressure(s) and/or heart rate (based on artificial intelligence (e.g., machine learning) according to an exemplary embodiment of the disclosed concept;

FIG. 18 is a block diagram showing the internal components of biometric sensor unit forming part of the system of FIG. 12 according to one non-limiting exemplary embodiment of the disclosed concept;

FIG. 19 is a block diagram of care provider computer system forming part of the system of FIG. 17 according to one non-limiting exemplary embodiment;

FIG. 20 is a schematic diagram of a system for monitoring and providing alerts of a fall risk by predicting risk of experiencing symptoms related to abnormal blood pressure(s) and/or heart rate (based on artificial intelligence (e.g., machine learning) according to an alternative exemplary embodiment of the disclosed concept;

FIG. 21 is a block diagram of a bedside biofeedback monitor according to an exemplary embodiment of the disclosed concept;

FIG. 22 is a schematic diagram of a system for monitoring and providing alerts of a fall risk by predicting risk of experiencing symptoms related to abnormal blood pressure(s) and/or heart rate (based on artificial intelligence (e.g., machine learning) according to yet another alternative exemplary embodiment of the disclosed concept;

FIG. 23 is a block diagram showing the internal components of a biometric sensor unit according to an alternative non-limiting exemplary embodiment;

FIG. 24 is a schematic diagram of an ear mounted biofeedback device according to one particular non-limiting exemplary embodiment of the disclosed concept;

FIG. 25 is a schematic diagram of a necklace biofeedback device according to another particular non-limiting exemplary embodiment of the disclosed concept;

FIG. 26 is a schematic diagram of a tie clip biofeedback device according to another particular non-limiting exemplary embodiment of the disclosed concept;

FIG. 27 is a schematic diagram of a pocket clip biofeedback device according to another particular non-limiting exemplary embodiment of the disclosed concept;

FIG. 28 illustrates the location of the dorsalis pedis and/or posterior tibial arteries in the lower extremities of the leg;

FIG. 29 illustrates pedal pulse sensor locations according to an aspect of the disclosed concept; and

FIGS. 30 and 31 show a pedal pulse sensor device implemented according to an exemplary embodiment of the disclosed concept.

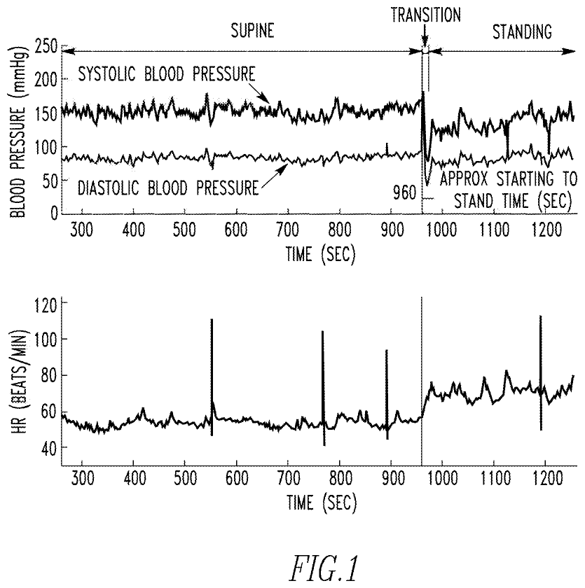

### DETAILED DESCRIPTION OF EXEMPLARY EMBODIMENTS

As used herein, the singular form of “a”, “an”, and “the” include plural references unless the context dictates otherwise.

As used herein, the statement that two or more parts or components are “coupled” shall mean that the parts are joined or operate together either directly or indirectly, i.e., through one or more intermediate parts or components, so long as a link occurs.

As used herein, the term “number” shall mean one or an integer greater than one (i.e., a plurality).

As used herein, the term “random forest” shall mean machine learning decision trees for classification and/or regression. The collection of the decision trees makes up the forest with larger number of decision trees yielding higher accuracy results unless the context dictates otherwise.

As used herein, the term “deep learning neural network” shall mean an artificial neural network with multiple hidden layers between the input and output layers that determines the correct mathematical manipulation (linear or non-linear) to turn the input into the output by moving through the layers and calculating the probability of each output unless the context dictates otherwise.

As used herein, the term “hidden layer” shall mean a neural network layer of one or more neurons whose output is connected to the inputs of other neurons and that, as a result, is not visible as a network output unless the context dictates otherwise.

As used herein, the term “recurrent neural network” shall mean a class of artificial neural network where connections between nodes form a directed graph along a temporal sequence and that therefore allows the network to exhibit temporal dynamic behavior unless the context dictates otherwise.

As used herein, the term “recursive deep learning neural network” shall mean a deep learning neural network that is also a recurrent neural network unless the context dictates otherwise.

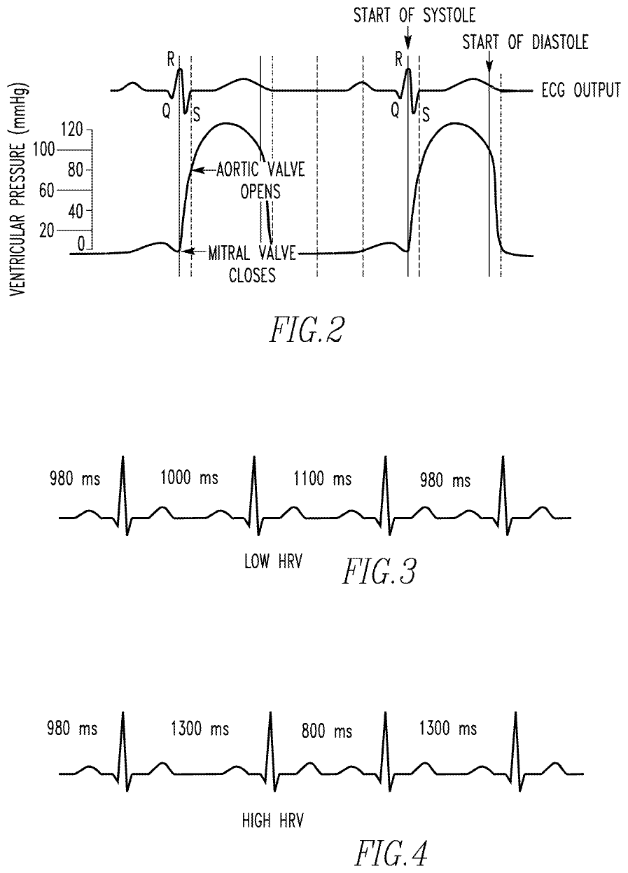

As used herein, the term “heart rate variability (HRV)” shall mean the variation in the time interval between consecutive heartbeats. In an ECG wave signal as shown in FIG. 2, the interval between heartbeats can be defined by two consecutive points such as QQ, RR, or SS interval. For illustrative purposes, RR interval is used as a non-limiting exemplary implementation, where a low HRV as shown in FIG. 3 indicates small variations in the RR intervals and a high HRV as shown in FIG. 3 indicates larger variations in the RR intervals. Methods used to detect RR intervals for determining HRV include, without limitation, an electrocardiogram (ECG or EKG) and the pulse wave signal derived from a photoplethysmograph (PPG). There is a direct correlation between ECG and arterial blood pressure wave-forms as shown FIG. 5. Thus, HRV can also be indirectly derived using continuous blood pressure.

As used herein, the term “HRV parameter” shall refer to statistical values derived from HRV data (e.g., RR intervals (although QQ and TT could also be used)), such as, but not limited to, averages or standard deviations. These HRV parameters shall include, without limitation, measures of HRV obtained using time-domain methods, frequency-domain methods, or nonlinear methods (e.g., a Poincaré plot).

As used herein, the term “low frequency (LF) range” shall mean from 0.04 to 0.15 Hz.

As used herein, the term “high frequency (HF) range” shall mean from 0.15 to 0.4 Hz.

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Directional phrases used herein, such as, for example and without limitation, top, bottom, left, right, upper, lower, front, back, and derivatives thereof, relate to the normal sense of the words.

The disclosed concept will now be described, for purposes of explanation, in connection with numerous specific details in order to provide a thorough understanding of the subject innovation. It will be evident, however, that the disclosed concept can be practiced without these specific details without departing from the spirit and scope of this innovation.

As described in greater detail herein in connection with particular exemplary embodiments, the disclosed concept deviates from current clinical practice of comparing pre- and post-orthostasis blood pressure and/or heart rate data to determine one's propensity of experiencing symptoms related abnormal blood pressures and/or heart rate upon orthostasis, such as, without limitation, OH symptoms.

In the disclosed concept, real-time heartbeat data, such as ECG QRS wave-form data or PPG sensor data, obtained during periods of low physical activity as indicated by physical motion sensors is extracted and selected features (HRV parameters) are analyzed by an artificial intelligence system. In one particular embodiment, both HRV parameters and the ECG QRS wave-form or PPG sensor data may be analyzed in combination to increase accuracy. Real-time may refer to the time period immediately before the person rises as detected by an accelerometer or other device for about the past 1 second to 1 hour, and preferably the past 2 seconds to 5 minutes.

The disclosed concept is able to determine and predict if an individual's ANS system is in a compromised state and unable to cope with the stress of standing. This is accomplished by using a device to detect when the individual is vertically rising, a heart rate monitor and an artificial based system. The accelerometer is discussed further below and is optional. The heart rate monitor is also discussed further below. The artificial intelligence (e.g., machine learning) based system may utilize, for example, an artificial neural network or random forest system, for detecting and providing alerts of potential symptoms related to abnormal blood pressure(s) and/or heart rate based on the real time monitoring of certain predetermined HRV parameters obtained from data collected by an ECG sensor, a PPG sensor, or other similar sensor device. In particular, as described in more detail herein, an artificial intelligence based system is trained to examine temporal changes in certain HRV parameters (determined from heartbeat data obtained before the individual stands up—e.g., as identified by certain physical motion sensors) in order to predict from such parameters and the temporal change thereof the risk that an individual will experience symptoms related to abnormal blood pressure(s) and/or heart rate if he or she stands. Temporal may mean past 1 second to 10 minutes, and preferably past 2 seconds to 5 minutes. The heartbeat data may be obtained by the person wearing a heart rate monitor. As an aspect of the disclosed concept, and as described in more detail herein, HRV parameters from human subject ECG or PPG test data are labeled with classifications (such as, but not limited to, whether or not abnormal blood pressure(s) and/or heart rate were present) and are used to train the artificial intelligence system. Specifically, the artificial intelligence system is trained so as to establish a set of baseline fall risk criteria and provide real-time prediction of fall risk episodes based on certain temporal changes of HRV parameter inputs. In addition, in a further aspect of the disclosed concept, the artificial intelligence system may be further trained to establish customized risk criteria for a particular individual using additional test data obtained specifically from that individual during one or more subsequent training phases.

Thus, in short, parameters established from, for example, ECG QRS wave-form make up the training data and are used to train an artificial intelligence algorithm to recognize abnormal blood pressure(s) and/or heart rate risks (e.g., OH risks) and/or intentions of standing. As discussed, the training data has been labeled with known classifications (e.g. FALLRISK, NO_FALLRISK, indicated by heart rate, systolic and diastolic blood pressures) and the supervised artificial intelligence (e.g., machine learning) algorithm learns to make predictions on the FALLRISK or NO_FALLRISK from new input data. In the exemplary embodiment, time series data (e.g., any HRV parameter versus time) is provided in short (e.g., 1 second) windows, although smaller or larger time windows can be used. Multiple parameters are combined, creating a rich time series that can be used by the algorithm to determine the best predictive model. Classifications of FALLRISK or NO_FALLRISK can be made using various statistical models (regression, Naïve Bayes, or Bayesian Networks) or using structural models that are rule based (Decision Trees, Random Forest), distance based (k-Nearest Neighbor, learning vector quantization), and Neural Networks (Multi-Layer Perceptron). In the exemplary embodiment, the Random Forest model is used, which implements ensemble theory to create a collection of decision tree (i.e., a forest) classifiers using randomly selected subsets of the training data. The model selects the best class to yield the highest predictive accuracy.

The disclosed concept is able to predict in advance if the person will be at FALLRISK or NO_FALLRISK (e.g., between 5 to 30 seconds in advance of it happening). Thus, care staff can opt to receive notifications as soon as the person is at risk. Another option is to allow the algorithm to send notifications as soon as the person has intentions to stand. Frail older adults typically require 15 to 30 seconds to transition from lying to standing. It is therefore anticipated that the disclosed concept will be used by frail older adults under professional care, and thus the ability to predict 5 to 30 seconds in advance is significant and allows the care staff to administer aide to the older adult in a timely fashion.

Thus, according to an aspect of the disclosed concept, described in greater detail herein in connection with a number of particular exemplary embodiments, a biometric sensor unit, such as a wearable health tracker, may be used to determine a person's RR interval (i.e., detect heartbeat data by, for example, single or multiple lead ECG methods). The biometric sensor unit can, for example, and without limitation, be worn on the chest as a strap, be worn on the wrist as a health watch, be worn in the ear, be integrated into a piece of clothing, or even be implanted in the body (essentially anywhere a pulse can be detected). The RR (or NN) intervals are then used to determine certain HRV parameter inputs, which inputs are then (without concurrently measured blood pressure data) fed to and analyzed by the trained artificial intelligence system to determine whether certain criterion for fall risks have been met. The inputs can be continuous or intermittent (such as every 1 seconds or 3 seconds or 10 seconds or 30 seconds). In one embodiment, transmission from the biometric sensor unit is initiated when there is physical motion as indicated by a physical motion sensor to conserve battery life of the biometric sensor unit. The artificial intelligence system can process entire streams of data, but to increase the artificial intelligence system's computation speed, analysis can be completed on selective time instances where a physical motion sensor indicates low activity (i.e. lying, sitting, or standing) when an individual is at the highest risk of experiencing symptoms related to abnormal blood pressure and/or heart rate. If a determination is made that certain criterion for fall risk have been met, a real-time alert (controlled by the continual assessment of the individual's fall risk as described herein), such as a cell phone vibration, a sound, an image, and or a video, is triggered and provided to the individual. This alert provides actionable cues that serve as reminders for the individual to flex their ankles back and forth to manually pump blood in their lower extremities or to pause and refrain postural change until there is no fall risk as determined by the trained artificial intelligence system. A different alert may also be provided to a family member or care provider, notifying them of the individual's fall risk should they standup, so that preemptive actions can be taken. The real-time alert will be terminated once it is safe for the individual to resume changes in position as determined by the trained algorithm. In some embodiments, described in greater detail herein, the real-time detection of a change in body position is used in combination with the determination that certain criterion for fall risk have been met to cause the alert to be triggered.

As stated elsewhere herein, HRV parameters may be time-domain based, frequency-domain based and/or non-linear based, and many such parameters may be employed in connection with the implementation of the disclosed concept. Thus, before describing particular embodiments of the disclosed concept in detail, a description of various suitable HRV parameters that may be employed in the disclosed concept will first be provided.

Typical time-domain HRV parameters are derived from the RR or NN intervals of collected heartbeat data (e.g., ECG data). A number of such time-domain HRV parameters are shown in Table 1 below (each of which is a direct and indirect measure of RR or NN distribution).

TABLE 1

| Parameter | Unit | Description |
|---|---|---|
| SDNN | ms | Standard deviation of NN intervals |
| SDRR | ms | Standard deviation of RR intervals |
| SDANN | ms | Standard deviation of the average NN intervals |
| | | for each 5 min segment of a 24 h HRV recording |
| SDNN index | ms | Mean of the standard deviations of all the NN |
| (SDNNI) | | intervals for each 5 min segment of a 24 h HRV |
| | | recording |
| pNN50 | % | Percentage of successive RR intervals that differ |
| | | by more than 50 ms |
| HR Max − HR | bpm | Average difference between the highest and |
| Min | | lowest heart rates during each respiratory cycle |
| RMSSD | ms | Root mean square of successive RR interval |
| | | differences |
| HRV triangular | N/A | Integral of the density of the RR interval |
| index | | histogram divided by its height |
| TINN | ms | Baseline width of the RR interval histogram |

As described in more detail herein, exemplary embodiments of the disclosed concept use one or more of these time-domain HRV parameters. It will be appreciated, however, that this is meant to be exemplary only, and that other time-domain HRV parameters (including those listed in Table 1 or additional parameters not listed in Table 1) may also be used within the scope of the disclosed concept. In one particular exemplary embodiment, three particular time-domain HRV parameters are used. Those three time-domain HRV parameters are pNN50, which represents the percentage of successive RR (or NN) intervals that differ by more than 50 ms, RMSSD, which represents the root mean square of successive RR (or NN) interval differences, and TINN, which is the baseline width of the RR (or NN) interval histogram. These variables are directly related to the short-term variation of the RR (or NN) intervals and corresponding high frequency content of the RR (or NN) intervals, and thus are believed to be particularly suited for providing information relating to the risk for experiencing symptoms related to abnormal blood pressure and/or heart rate and the ability to properly respond thereto.

Any time domain curve, in this case the RR interval versus time curve, can be represented as a sum of multiple sinusoidal oscillations at different amplitudes and frequencies between 0.0033 Hz to 0.4 Hz. The process of analyzing a time domain curve using frequencies is called the Fast Fourier Transform (FFT). Results of an FFT provide information on the frequency content of the curve and associated power of those frequencies.

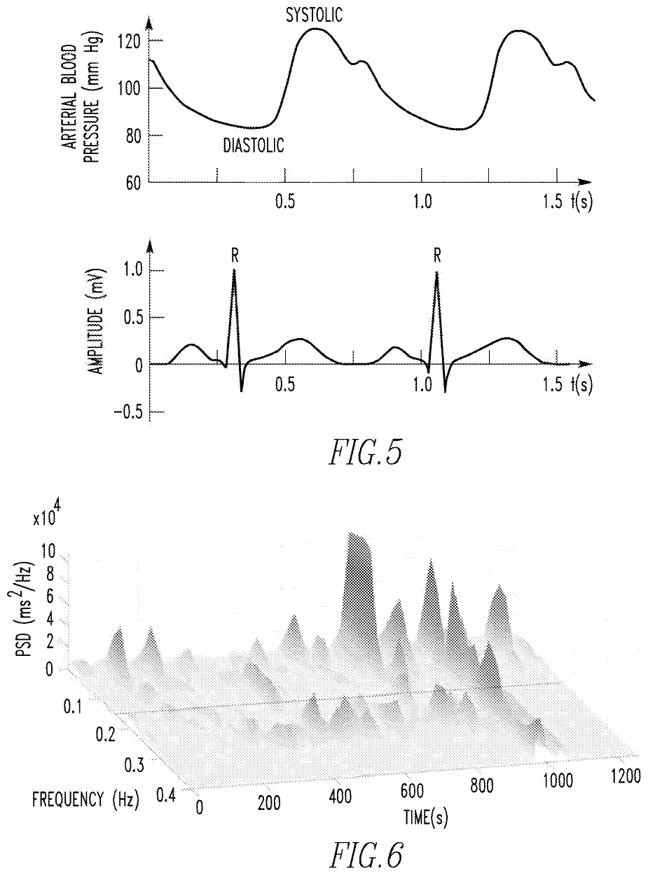

The frequency bands are typically ultra-low frequency (ULF) from 0 to 0.0033 Hz, very low frequency (VLF) from 0.0033 to 0.04 Hz, low frequency (LF) from 0.04 to 0.15 Hz, and high frequency (HF) from 0.15 to 0.4 Hz. These bands are further divided into smaller bins, yielding finer details about the presence and power of these frequencies in the original RR interval versus time curve. The time varying nature of the frequency content of an individual's RR intervals can best be visualized using a 3D frequency spectrogram as shown in FIG. 6. Of interest to the disclosed concept are the LF and HF frequency ranges as they are more relevant for RR or NN intervals investigated in relative short periods of time (e.g., seconds to minutes range).

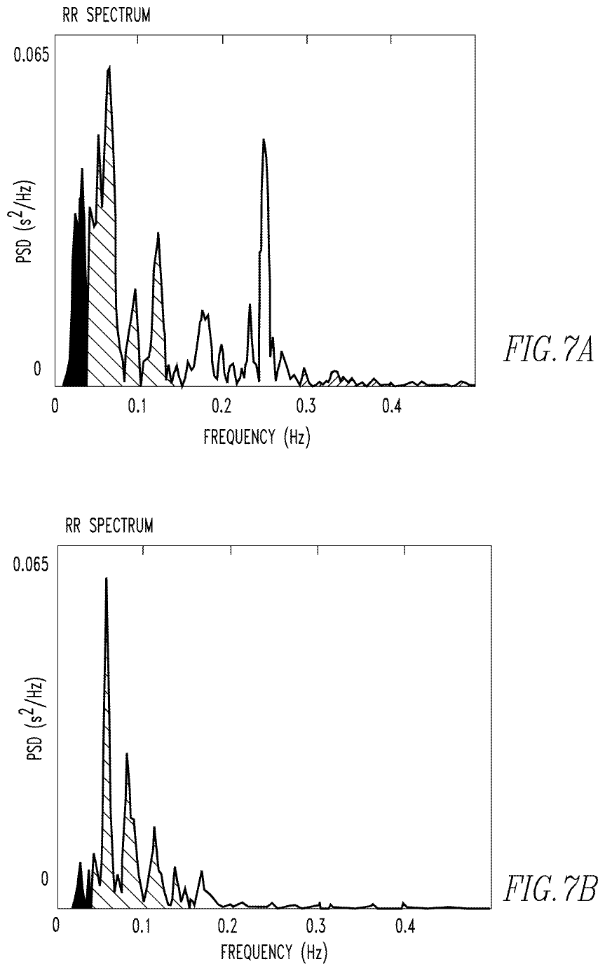

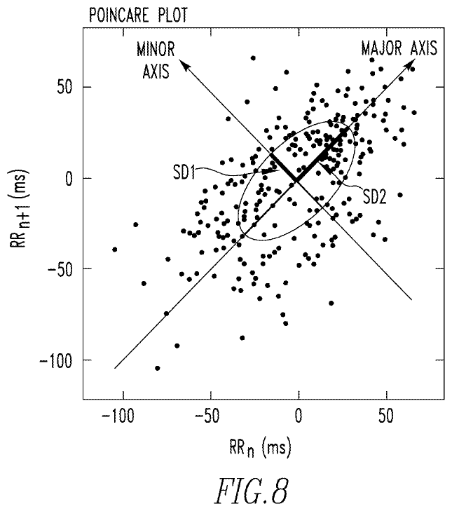

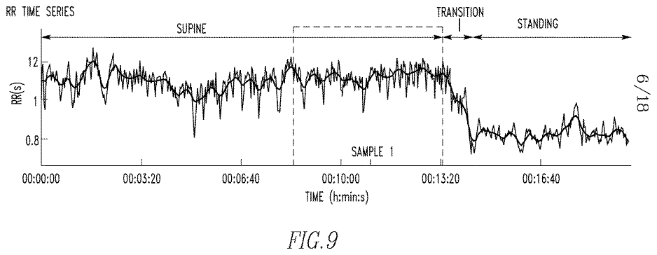

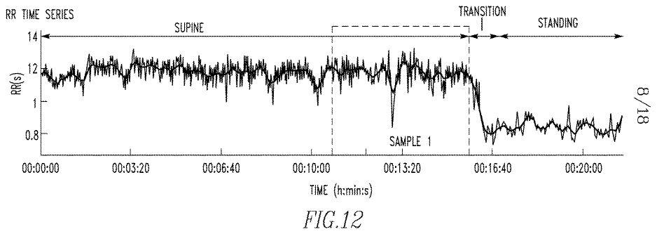

FIGS. 7A and 7B show exemplary PSD graphs from an individual. FIG. 7A is generated using the 5-minute windows labeled SAMPLE 1 of FIG. 9, exhibiting high PSD in both the LF and HF ranges. FIG. 7B is generated using the 5-minute window labeled SAMPLE 1 of FIG. 12, but only shows high PSD in the LF range. One can envision similar PSD vs. Frequency graphs with shorter time windows of 1 second, or 3 seconds, or 5 seconds, or 1 minute, for example. Peaks (i.e., high PSD level) at approximately 0.25 Hz in the HF range are present in FIG. 7A (but minimal in FIG. 7B) and indicate proper PNS function. Peaks in the LF range (hatched regions) as seen in both FIG. 7A and FIG. 7B indicate functioning of the interplay of both the SNS and PNS branches with the SNS being the dominant player. It is known that the PNS branch of the ANS is responsible for vagal tone (i.e. activity such as heart rate, dilation and constriction of vessels that affect blood pressure.) Furthermore, presence of HF peaks is a measure of the individual's vagal modulation of heart rate as it responds to changing workloads (such as from transition from lying to standing). Thus, absence or weak presence of peaks in the HF range represents overall poor PNS function (i.e. poor vagal modulation of heart rate), placing the person at risk for experiencing symptoms related to abnormal blood pressure and/or heart rate. Accordingly, one exemplary embodiment of the disclosed concept uses PSD in the HF range as one of the HRV parameters. It will be appreciated, however, that this is meant to be exemplary only, and that other frequency-domain HRV parameters may also be used within the scope of the disclosed concept.

In the exemplary embodiment, non-linear based domain analysis consists of creating a scatter plot called a Poincaré plot. In a Poincaré plot, the x-axis represents the RRₙ (or NNₙ) value with the subsequent RRₙ₊₁ (or NNₙ₊₁) value being plotted on the y-axis. For a given time window, a cluster of data points can be fitted with a rotated ellipse as shown in the exemplary Poincaré plot of FIG. 8. The major axis perpendicular to the minor diameter of the Poincaré ellipse is called the line of identity and is represented by the equation y=x or RRₙ=RRₙ₊₁ (or NNₙ=NNₙ₊₁). As seen in FIG. 8, the minor axis is along the minor diameter of the ellipse and is perpendicular to the line of identity. The standard deviation of the nominal value of the minor axis is referred to herein as SD1 and represents the short-term variation of RR (or NN). The standard deviation of the Poincaré plot along the major axis is referred to herein as SD2. The area of the ellipse represents total HRV and correlates to baroreflex sensitivity, LF and HF power, and RMSSD. SD1 correlates to short-term changes in RR (or NN) intervals and is directly related to PNS activity and performance. SD2 correlates to long-term changes of the RR (or NN) interval and is directly related to how well the PNS and ANS work together with the SNS as the dominant player in this relationship. A Poincaré plot generated from collected heartbeat data (e.g., based on ECG signals), or one or more parts thereof, are thus used as the HRV parameter(s) in one exemplary embodiment of the disclosed concept. It will be appreciated, however, that the use of a Poincaré plot, or one or more parts thereof, as the HRV parameter(s) is meant to be exemplary only, and that other non-linear based HRV parameters may also be used within the scope of the disclosed concept.

A number of particular examples of HRV parameters in accordance with the disclosed concept will now be discussed in connection with FIGS. 9-14 to aid in understanding how such data may be useful in predicting the risk of experiencing symptoms related to abnormal blood pressure and/or heart rate. In particular, the examples shown in FIGS. 9-14 show data for an individual collected during two test periods (Test 1 and Test 2).

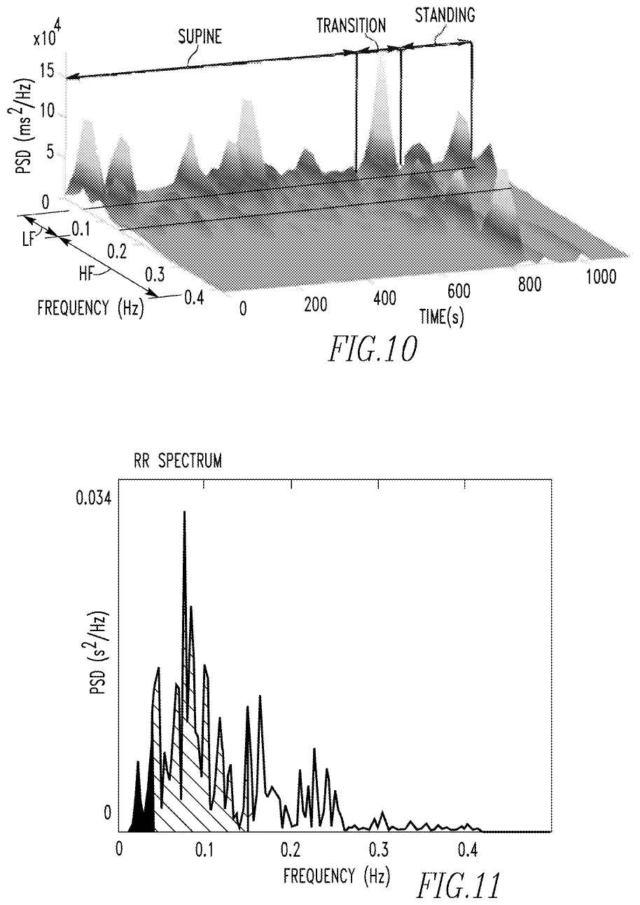

FIG. 9 shows RR vs. time while an individual is in a supine position (00:00:00 to 00:13 m: 43 s), in transition from lying to sitting (00:13 m: 27 s to 00:14 m: 27 s), and then standing (00:14 m: 27 s to 00:19:38 s) during the test period of Test 1. Note that the RR interval while in supine has an average of 1.1 seconds and then quickly decreases to approximately 0.8 seconds when the person begins to stand up. The decreased RR values indicate that the heart is beating faster to cope with the stress of standing resulting in blood that was pooled in the lower extremities and the need to get blood back to the heart and brain. In FIG. 10, which is a 3D frequency spectrogram as a function of time for the same individual, the y-axis is divided into three frequency ranges, very low frequency (0.00 to 0.04 Hz), low frequency (0.04 to 0.15 Hz), and high frequency (0.15 to 0.4 Hz). The z-axis represents PSD, where peaks and valleys represent high and low powers. The x-axis represents time. It is evident from FIG. 10 that there are a significant number of peaks as well as high power in the LF range in comparison to the HF range from approximately 0 to 600 seconds. Absence of peak in the HF range within this time period indicates that this person's PNS activity is in a compromised state and, as a result, this person is at risk for experiencing symptoms related to abnormal blood pressure and/or heart rate (which was indeed the case). This difference can also be observed by the power spectral density of FIG. 11 for the 5-minute window labeled SAMPLE 1 in FIG. 9.

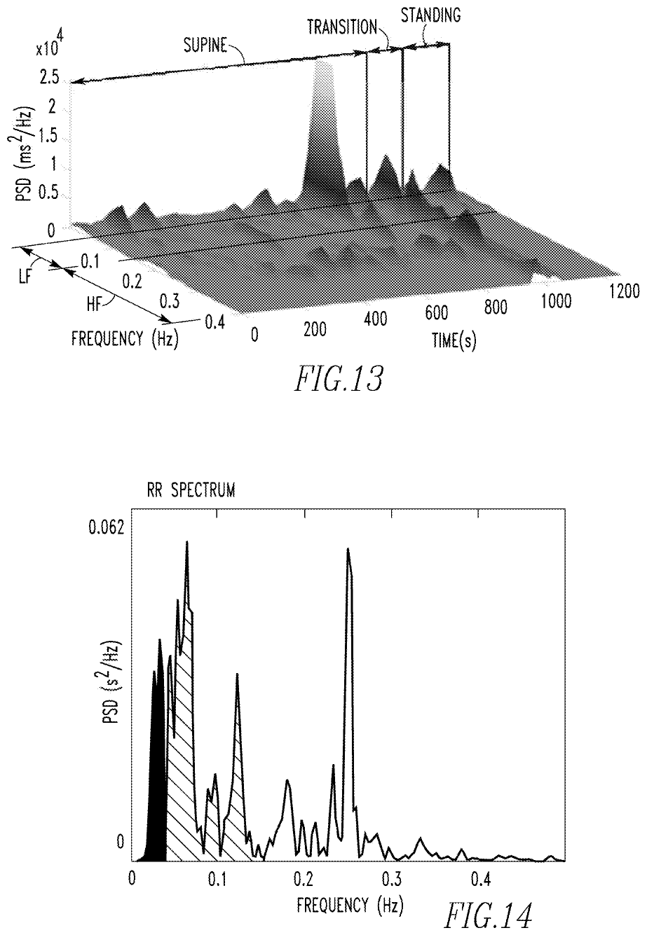

After Test 1 was completed (900 seconds in supine (only 600 seconds is shown) and 300 seconds in standing position), the same individual conducted Test 2 (immediately after Test 1 without interruption). FIG. 12 shows RR vs. time from 00:00 m: 00 s to 00:21 m: 31 s. The average RR value prior to standing is 1.2 seconds and decreases to approximately 0.8 seconds upon standing. As seen in the spectrogram of FIG. 13, there is minimal presence of HF peaks from 0 to approximately 300 seconds. From 300 seconds, peaks in both LF and HF are noticeable, which is indicative of healthy SNS and PNS activities, respectively. Furthermore, peaks in the HF range indicate that the individual's vagal modulation of heart rate is possible and that this person was not in danger of experiencing symptoms related to abnormal blood pressure and/or heart rate (which was indeed the outcome). This difference can also be observed by the PSD vs. Frequency graph shown in FIG. 14 for this same individual in Test 2 generated using the 5-minute window labeled SAMPLE I in FIG. 12.

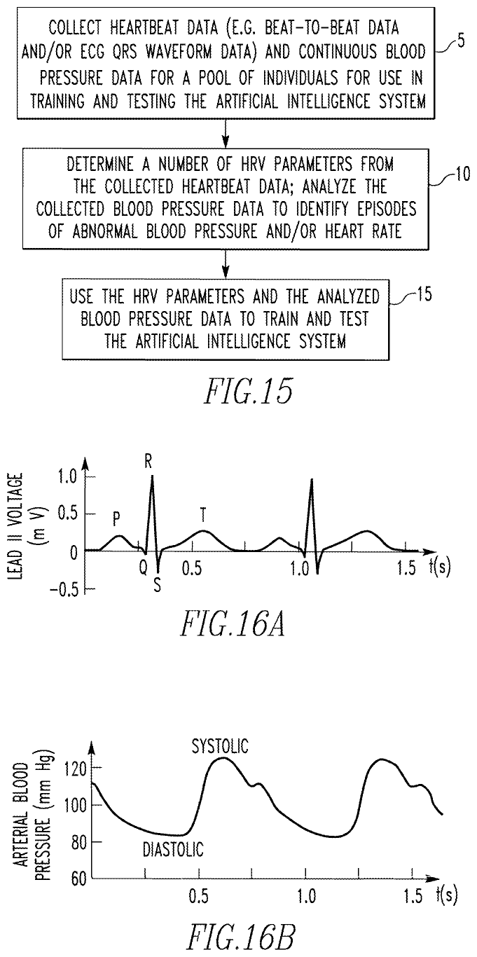

FIG. 15 is a flowchart that illustrates a method of training and testing an artificial intelligence system (e.g. a machine learning system) according to an exemplary embodiment of the disclosed concept. In one particular embodiment, the artificial intelligence system that is trained and tested in FIG. 15 is an artificial neural network, such as a deep learning neural network, and, more specifically, a recursive deep learning neural network. In another particular embodiment, the artificial intelligence system that is trained and tested in FIG. 15 is a random forest system.

Referring to FIG. 15, the method begins at step 5, wherein heartbeat data (e.g., beat-to-beat data and/or ECG QRS waveform data) and continuous blood pressure data is collected as a function of time for a large number of individuals for use in training and testing the artificial intelligence system. Typical ECG and blood pressure signals are as shown in FIGS. 16A and 16B, respectively. In the exemplary embodiment, heartbeat data (e.g., from an ECG monitor) and blood pressure data (e.g., from a finger and/or brachial blood pressure cuff) are collected for the large number of individuals during a number of orthostatic stand tests that include alternating periods wherein the patients are lying down and standing up. In one particular exemplary embodiment implemented by the present inventors, such data was collected for the individuals during three active orthostatic stand tests. In such particular exemplary embodiment, each individual wore a finger arterial blood pressure cuff, a brachial blood pressure cuff, a 5-lead ECG II monitor, and a separate 1-lead ECG monitor. The finger arterial blood pressure cuff, brachial blood pressure cuff, and 5-lead ECG II monitor acquired data at 300 Hz, and the 1-lead ECG monitor collected data at 1000 Hz. It will be understood, however, that this is meant to be exemplary only, and that wearing two ECG monitors is not necessary, as ultimately the disclosed concept only requires a minimum of 1-lead ECG (although more leads can be used). Two ECG monitors may be used to allow one to serve as a back-up just in case one of the ECG monitors does not acquire data properly.

During each of the orthostatic stand tests, the individual first lies supine for 5 minutes to bring his/her body to a state of rest before each test. Then, the individual lies supine for 10 minutes, gets up from the table, and stands for 5 minutes. In this particular exemplary embodiment, this is done three times without interruption. Note, however, that this is meant to be exemplary only, and that other protocols for collecting data may also the used. For example, the same test can be conducted while sitting for 10 minutes and then standing for 5 minutes should the individual be limited in his/her ability to conduct the maneuver independently.

Next, at step 10, a number of HRV parameters (e.g., time-domain based, frequency-domain based and/or non-linear based parameters) are determined from the collected heartbeat data. Also, the collected blood pressure data is analyzed to identify abnormal blood pressure and/or heart rate by detecting sustained blood pressure drops or heart rate increases as described herein. Then, the analyzed blood pressure data is used to tag the HRV parameter data. More specifically, in one particular exemplary embodiment, when there is a systolic blood pressure drop(S), diastolic blood pressure drop (D), or increase in heart rate (H) that meets the definition of an abnormal blood pressure and/or heart rate episode (such as, without limitation, an OH episode), HRV parameter data for 0 to 30 seconds prior to standing up (or could be in the 30 seconds to minutes range, depending upon training results) is classified with an identifier. For example, if systolic blood pressure dropped, diastolic blood pressure did not drop, and heart rate increased, then the HRV parameter data prior to standing up is classified as FALLRISK SH. If there are no signs of abnormal blood pressure and/or heart rate, then data prior to standing is tagged as NO FALLRISK. Thus, following step 10, data will have been assembled for each of the conducted orthostatic stand tests that includes classified or labelled HRV parameters for the time 0 to 30 seconds prior to standing up (or could be in the 30 seconds to minutes range, depending upon training results).

In one particular exemplary embodiment, the only HRV parameters that are determined at step 10 are frequency-domain based and comprise PSDs in the HF range or the LF and HF ranges. In another, alternative particular exemplary embodiment, time-domain based, frequency-domain based and/or non-linear based HRV parameters are determined at step 10 and include one or any combination of pNN50, RMSSD, and TINN as time-domain based HRV parameters, PSDs in the HF range or the LF and HF ranges as frequency-domain based HRV parameters, and Poincaré plots and/or or one or more parts thereof (e.g., the SD1 and SD2 values as described herein) as non-linear based HRV parameters. Other suitable parameters include average duration and rate of change of Q to R and R to S of the ECG QRS waveform. The QRS waveform represents the electrical signal of the heart as the ventricles begin to contract (point Q), where the bulk of the ventricle muscles completes its contraction (point R) and marks the beginning of systole. That is, the start of the heart pumping blood into the arteries. There are smaller areas of the ventricle that contract and complete the full contraction cycle, and are identified by point S. Again, it will be understood that these particular embodiments are meant to be exemplary only, and that other implementations using different sets of parameters from the QRS waveform may also be used within the scope of the disclosed concept. For example, and without limitation, the following parameters in any combination may be used alone or in combination with the particular embodiments just described: PNS index (which is a function of mean RR, RMSSD, and SD1) and SNS index (which is a parameter based upon mean heart rate, Baevsky's stress index and SD2), average duration and rate of change of Q to R and/or R to S, QQ intervals, RR intervals, SS intervals, and heart rate.

Next, at step 15, the determined one or more parameters derived from the QRS waveform and the analyzed (e.g., labeled) blood pressure data are used to train and test the artificial intelligence system. Specifically, the artificial intelligence system, which is a machine learning system (e.g., artificial neural network) in the exemplary embodiment, is trained to be able to predict abnormal blood pressure and/or heart rate episodes (and, in particular, when an individual's ability to respond to orthostatic stress is compromised) based on the selected HRV parameter inputs, and in particular based on the monitoring of temporal changes in those HRV parameter inputs. As a result, the trained artificial intelligence system will establish and be able to detect a set of baseline criteria for predicting abnormal blood pressure and/or heart rate episodes based on certain QRS waveform parameter inputs. As noted elsewhere herein, in an alternative exemplary embodiment, once deployed, the artificial intelligence system may thereafter be further trained with data specific to a particular individual in question by collecting additional heartbeat and blood pressure data from that individual during one or more subsequent training phases, and using that collected data to further train the artificial intelligence system so as to be customized to that particular individual.

Thus, following step 15, an artificial intelligence system is provided that is able to predict when an individual's ability to respond to orthostatic stress is compromised. In other words, the trained artificial intelligence system has the ability to recognize and predict when a person's autonomic nervous is incapable of coping with orthostatic stress leading the person to experience symptoms related to abnormal blood pressure and/or heart rate. As discussed elsewhere herein, these symptoms include, but are not limited to, light-headedness, visual blurring, dizziness, generalized weakness, fatigue, cognitive slowing, leg buckling, coat-hanger ache, and gradual or sudden loss of consciousness (i.e. syncope). These symptoms place the individual at a high risk of an injurious fall. As just described, this is accomplished by analyzing temporal changes in the parameters derived from heartbeat (e.g., ECG) data, specifically the RR or NN interval data, in the time, frequency, and/or nonlinear domains.

FIG. 17 is a schematic diagram of a system 20 for monitoring fall risk by predicting risk for experiencing symptoms related to abnormal blood pressure and/or heart rate based on machine learning according to an exemplary embodiment of the disclosed concept. As seen in FIG. 17, system 20 comprises a plurality of components including a biometric sensor unit 25, a receiver unit 30 in proximity to and in electronic communication with biometric sensor unit 25, a network 35, a central computer system 40 including a predictive artificial intelligence (AI) system 45, and a care provider computer system 50. Each of these components is described in detail below. As seen in FIG. 17, receiver unit 30, central computer system 40, and care provider computer system 50 are all in electronic communication with network 35 to facilitate operation of system 20 as described herein. Furthermore, while in the illustrated exemplary embodiment the AI system resides in the “cloud”, it will be understood that is may also implemented locally on a computing device such as a PC.

Biometric sensor unit 25 is structured and configured to be worn by an individual to be monitored. For example, biometric sensor unit 25 may be worn by an individual at a hospital, nursing home, or any other location where the individual might be at a risk of falling and therefore needs to be monitored.

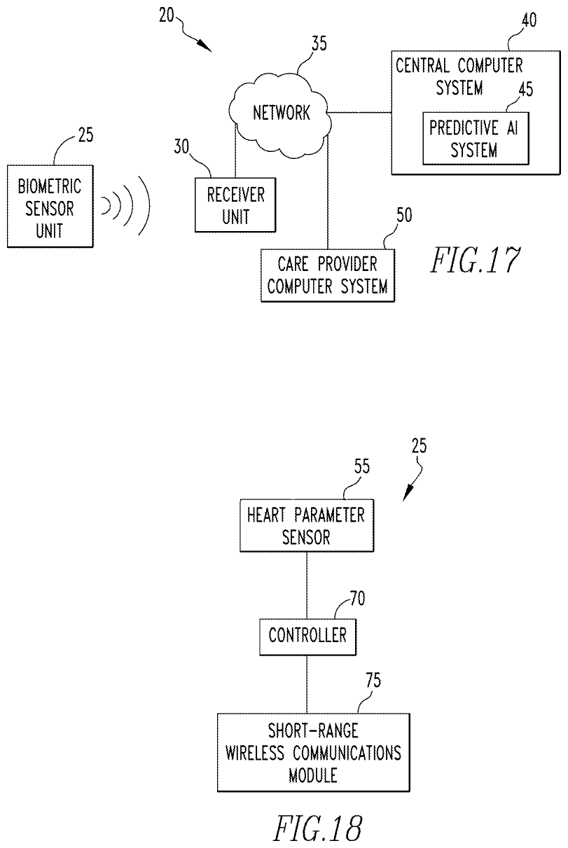

FIG. 18 is a block diagram showing the internal components of biometric sensor unit 25 according to one non-limiting exemplary embodiment. The exemplary biometric sensor unit 25 includes a heart parameter sensor 55 structured and configured to determine heartbeat data for the individual wearing biometric sensor unit 25. In the exemplary embodiment, heart parameter sensor 55 is an ECG sensor (e.g., 1 to 12 leads), although it will be appreciated that other types of heart parameter sensors (such as a PPG sensor) may also be employed within the scope of the disclosed concept. In one embodiment, heart parameter sensor 55 is capable of measuring an interbeat interval of at least 100 hertz. In another embodiment, heart parameter sensor 55 is capable of measuring an interbeat interval of at least 300 hertz,

Biometric sensor unit 25 further includes a controller 70 coupled to receive the outputs of heart parameter sensor 55. Finally, wearable biometric sensor unit 25 includes a short-range wireless communications module 75 that is structured and configured to enable wearable biometric sensor unit 25 to communicate with receiver unit 30 over a short-range wireless network. Short-range wireless communications module 75 may be, for example and without limitation, a WiFi module, a Bluetooth® module, a ZigBee module, an IEEE802.15.4 module, or any other suitable short-range wireless communications module that provides compatible communications capabilities.

Referring again to FIG. 17, in the exemplary embodiment, receiver unit 30 is a computing device which may be, for example and without limitation, a smartphone, a tablet PC, a laptop, or some other portable computing device. Receiver unit 30 may also be a non-portable computing device such as a desktop PC. Receiver unit 30 is structured to be able to communicate wirelessly with biometric sensor unit 25 over the short-range wireless network as described above. In addition, receiver unit 30 is structured and configured to be able to communicate with network 35 by way of a wired or wireless connection. In the exemplary embodiment, receiver unit 30 stores and implements a software application (e.g., a web/mobile app) that allows it to collect and transmit data as described herein.

Network 35 may be, for example, the Internet, one or more private communications networks, or any combination thereof. As employed herein, the term “communications network” shall expressly include, but not be limited by, any local area network (LAN), wide area network (WAN), intranet, extranet, global communication network, the Internet, and/or wireless communication network. Preferably, the wired and/or wireless connections to network 35 are secure (e.g., in the form of an encrypted virtual private network).

Central computer system 40 comprises any suitable processing or computing system having a computing device and one or more memory components for data storage (e.g., a controller), such as, without limitation, one or more PCs or server computers. As seen in FIG. 17, central computer system 40 houses and implements a component comprising predictive artificial intelligence (AI) system 45. More specifically, central computer system 40 has stored therein a number of routines that are executable by controller and that implement (by way of computer/processor executable instructions stored on a tangible medium) at least one embodiment of the trained artificial intelligence system (e.g., trained by the method of FIG. 15) as described herein.

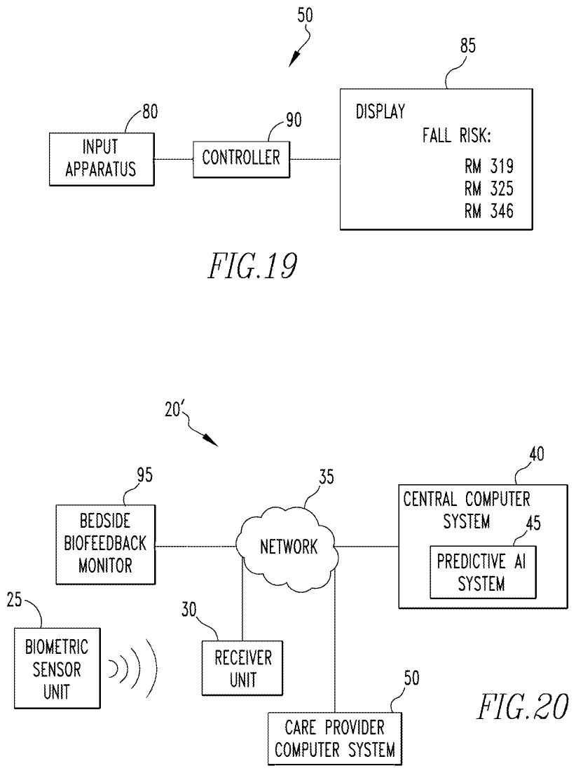

FIG. 19 is a block diagram of care provider computer system 50 according to one non-limiting exemplary embodiment. Care provider computer system 50 is associated with the facility in which the individual wearing biometric sensor unit 25 is being housed. The exemplary care provider computer system 50 includes an input apparatus 80 (such as a keypad or keyboard), a display 85 (such as an LCD or a touchscreen), and a controller 90. A user is able to provide input into controller 90 using input apparatus 80 (and/or display 85 if it is a touchscreen). Controller provides output signals to display 85 to enable display 85 to display information to the user as described in detail herein.

In operation, heart parameter sensor 55 of biometric sensor unit 25 continuously collects heartbeat data (e.g., ECG data) from the individual wearing biometric sensor unit 25 and transmits that data wirelessly to receiver unit 30 using short-range wireless communications module 75. In the exemplary embodiment, the collected heartbeat data is transmitted to receiver unit 30 in packets of time which can range from seconds to minutes. Receiver unit 30 in turn transmits the received heartbeat data to central computer system 40 through network 35 for analysis by predictive AI system 45 of central computer system 40. Note that in the present implementation, a blood pressure monitor is no longer needed once the algorithm of predictive AI system 45 has been trained. However, it could be used as a redundant system.

Predictive AI system 45 receives the heartbeat data from receiver unit 30 as just described and uses the trained artificial intelligence system of predictive AI system 45 to predict the likelihood of the onset of experiencing symptoms related to abnormal blood pressure and/or heart rate by checking the heartbeat data against the baseline criteria. More specifically, predictive AI system 45 determines from the received heartbeat data those particular HRV parameters that have been used to train the artificial intelligence system of predictive AI system 45 as described elsewhere herein. Predictive AI system 45, in particular the trained artificial intelligence system thereof, then analyzes the determined HRV parameters to determine the level of fall risk based on the baseline criteria. In the exemplary embodiment, the risk is calculated in terms of a percentage. If predictive AI system 45 determines that the criteria are met (e.g., if greater than a predetermined percentage risk is determined), central computer system 40 will generate and transmit a signal to alert a care provider (e.g. professional care staff, family, and/or loved ones) and the individual themselves in advance of the impending onset of experiencing symptoms related to abnormal blood pressure and/or heart rate so preemptive actions can be taken to mitigate potential injuries. In particular, in one embodiment, central computer system 40 generates and transmits a signal to care provider computer system 50 (facilitated through web applications) in order to provide advance notice of the fall risk associated with the impending onset of experiencing symptoms related to abnormal blood pressure and/or heart rate for the individual wearing biometric sensor unit 25. In the exemplary embodiment, display 85 of care provider computer system 50 will display information identifying the current fall risks at the location in question as shown in FIG. 19. In other embodiments, central computer system 40 can generate and transmit signals to the computers and/or phones of family/loved ones (again facilitated through web applications) to notify them of the risk and/or to receiver unit 30 in order to notify the individual being monitored of the risk directly.

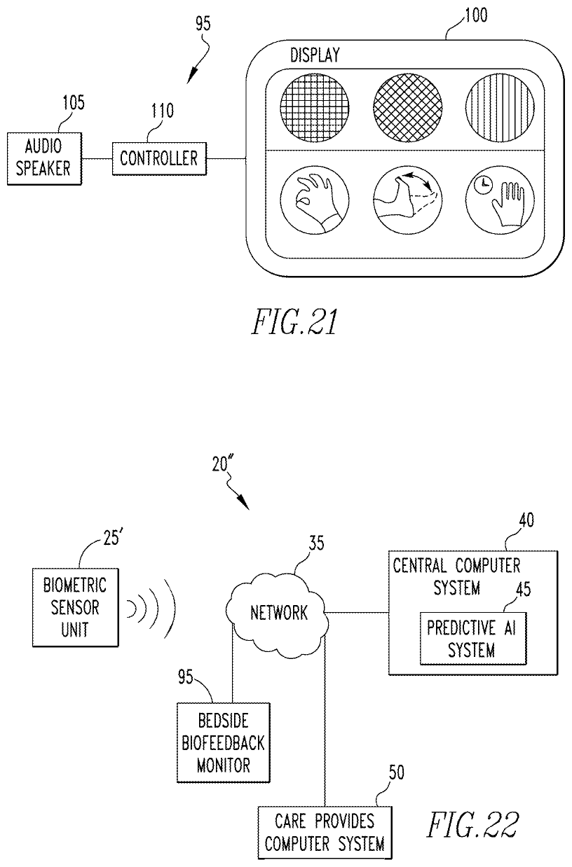

FIG. 20 is a schematic diagram of a system 20′ for monitoring fall risk by predicting risk for experiencing symptoms related to abnormal blood pressure and/or heart rate based on artificial intelligence (e.g., machine learning) according to an alternative exemplary embodiment of the disclosed concept. System 20′ is similar to system 20 shown in FIG. 17, and like components are labeled with like reference numerals. However, as seen in FIG. 20, system 20′ further includes a bedside feedback monitor 95 that is provided in the location where the individual wearing biometric sensor unit 25 resides. FIG. 21 is a block diagram of a bedside feedback monitor 95 according to an exemplary embodiment. As seen in FIG. 21, bedside biofeedback monitor 95 includes a display 100 (e.g., an LCD screen), an audio speaker 105, and a controller 110 to which display 100 and audio speaker 105 are connected and which controls the operation of those devices. The purpose of the biofeedback monitor is to provide the individual real-time cues to avoid experiencing symptoms related to abnormal blood pressure and/or heart rate. As the individual complies with the cues, their QRS waveform signals will change due to the response of the autonomic nervous system. If it changes in a manner that experiencing symptoms related to abnormal blood pressure and/or heart rate is reduced or eliminated, the individual will be provided new information on the biofeedback monitor indicating that it is okay to stand up. In addition, bedside biofeedback monitor 95 is in wired and/or wireless electronic communication with network 35 in order to receive real-time actionable cues on how to avoid experiencing symptoms related to abnormal blood pressure and/or heart rate. Specifically, bedside biofeedback monitor 95 receives information from central computer system 40 over network 35 with the purpose of communicating risk levels in real-time to the individual wearing wearable biometric sensor unit 25 as s/he complies with the actionable cues.

In the exemplary embodiment, based upon the risk level determined by predictive AI system 45, bedside biofeedback monitor 95 will output certain visual, tactile and/or audible information. For example, as shown in FIG. 21, display 100 can display a green light with an “OK” symbol to indicate that it is okay to stand up, a yellow light with a symbol prompting the individual to increase their blood circulation (such as by flexing their ankles back and forth) if the individual needs to take caution prior to standing, and a red light with a “WAIT” symbol if the individual should not stand up and instead wait for the care staff for assistance. Since predictive AI system 45 constantly analyzes the individual's heartbeat data, bedside biofeedback monitor 95 is able to display up-to-date status of the individual's risk for experiencing abnormal blood pressure and/or heart rate symptoms should they stand up. The biofeedback just described may also be provided to a user by way of receiver unit 30 in the embodiment of FIG. 17.

FIG. 22 is a schematic diagram of a system 20″ for monitoring fall risk by predicting risk for experiencing abnormal blood pressure and/or heart raterelated symptoms based on artificial intelligence (e.g., machine learning) according to another alternative exemplary embodiment of the disclosed concept. System 20″ is similar to system 20 shown in FIG. 17, and like components are labeled with like reference numerals. However, as seen in FIG. 22, system 20″ includes an alternative wearable biometric sensor unit 25′ that is similar to wearable biometric sensor unit 25, except that it includes a long-range wireless occasions module in order to enable it to communicate directly with network 35. In still a further alternative embodiment, a similar biometric sensor unit may be provided that, instead of having a wireless connection to network 35, has a wired connection.

In still a further alternative embodiment, to increase accuracy, the artificial intelligence system of predictive AI system 45 is trained with patient specific heartbeat data (e.g., ECG data) to establish individualized criteria for experiencing symptoms related to abnormal blood pressure and/or heart rate. More specifically, the artificial intelligence system of predictive AI system 45 in FIG. 17, 20 or 22 is further trained and tested with data specific to a particular individual collected by the biometric sensor unit during one or more subsequent training phases. As will be appreciated, the collected data will need to be classified or labeled as FALLRISK or NO_FALLRISK (along with S, D, and/or H descriptors) as described elsewhere herein. For that purpose, one or more continuous blood pressure sensors may be employed along with the biometric sensor unit in order to collect continuous blood pressure data for identifying episode of symptoms related to abnormal blood pressure and/or heart rate as described herein.

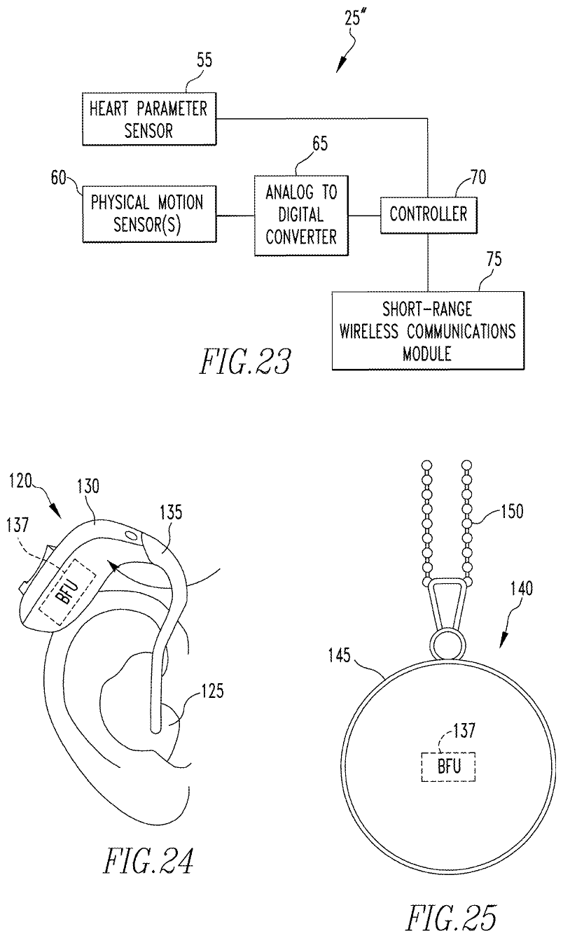

FIG. 23 is a block diagram showing the internal components of a biometric sensor unit 25″ according to an alternative non-limiting exemplary embodiment. The alternative exemplary biometric sensor unit 25″ is similar to biometric sensor unit 25, and like parts are labeled with like reference numerals. However, as seen in FIG. 23, biometric sensor unit 25″ further includes a number of physical motion sensors 60 structured and configured to measure one or more motion parameters of the individual wearing wearable biometric sensor unit 25″. An analog-to-digital converter 65 is coupled to the physical motion sensor(s) 60 in order to convert the analog signals sensed thereby into digital form before being provided to controller 70. Biometric sensor unit 25″ may be used in any of systems 20, 20′ or 20″ in order to enable such system to function as a physical intention predictor as described below for predicting the intention of the wearer of biometric sensor unit 25″ to transition from a lying position or a sitting position to an upright, standing position (this is when someone with risk of experiencing symptoms related to abnormal blood pressure and/or heart rate is in the most danger). In the exemplary embodiment, such intention is predicted for the purpose of enabling a notification to be provided to a care provider or other individual as described herein that the wearer of biometric sensor unit 25″ has intentions of standing, so that the care provider or other individual can take appropriate action. In one embodiment, the care provider or other individual can opt to receive such notifications of intention to stand even if there is no determined risk for experiencing symptoms related to abnormal blood pressure and/or heart rate as determined in the manner described in detail elsewhere herein.

More specifically, in this exemplary embodiment, physical motion parameters derived from data collected from a pool of test subjects (e.g., by way of sensor(s) similar to physical motion sensor(s) 60) are used to train predictive AI system 45 to be able to determine/predict when an individual has an intention of standing. Once so trained, predictive AI system 45 may thereafter be used to determine/predict when a wearer of biometric sensor unit 25″ has an intentions of standing based on data collected by physical motion sensors 60.

The particular physical motion parameters that may be used include, for example and without limitation, the rate of change, absolute magnitudes, or difference magnitudes in each axis direction of certain sensed data, and/or statistical descriptors of such data, such as mean, standard deviation, and variance of these parameters. In addition, in the exemplary embodiment, the training data includes data that is labeled with the start of specific body movements. Such labels may include, for example, lying on the left side, lying on the right side, lying on the back, or lying on stomach. In addition, the data is further labeled with the instant that physical position changes are made. Different physical maneuvers may also be identified. For example, such maneuver may include an individual maneuvering from lying to sitting by making a sit-up exercise motion, or maneuvering from lying to sitting by rolling onto their side (left or right), and then pushing off the bed to an upright sitting position.

Thus, in connection with the various embodiment described herein (including those employing an altimeter as described below), the motion data generated by physical motion sensors 60 will be sufficient to distinguish between resting data and vertical rising data, wherein the resting data represents when the subject is resting in the lying or sitting position and the vertical rising data represents when the subject is moving from a lying position to a sitting or standing position or from a sitting position to a standing position. In addition, the resting data and the vertical rising data will be synced to the HRV data to identify the portion of the HRV data that is representative of when the person is resting and the portion of the HRV data that is representative of when the person is vertically rising.

In addition, a low- and high-pass filter may be used to separate the sensed time varying signal into AC and DC components. The AC component is generally attributed to accelerations of the body and can be used to detect and predict intentions of standing. The DC component is the gravitational contribution and can also be used to classify body postures (lying, sitting, standing, etc.).

In the exemplary embodiment, predictive AI system 45 is first trained with data in order to establish a baseline set of criteria as described elsewhere herein. Thereafter, predictive AI system 45 may be further trained to establish individual-specific physical motion intentions using data from the individual using physical motion sensor(s) 60.

In one exemplary embodiment, physical motion sensor(s) 60 consist of a 3-axis accelerometer. In another exemplary embodiment, physical motion sensor(s) 60 consists of a 3-axis accelerometer and a 3-axis gyroscope. In still another exemplary embodiment, physical motion sensor(s) 60 consists of a 3-axis accelerometer, a 3-axis gyroscope, and a 3-axis magnetometer. In yet other embodiments, physical motion sensor(s) 60 comprise a laser beam, radar, and/or proximity sensor, in any combination and/or in combination with the sensor(s) described above or below.

In a minimized configuration, intentions of standing may be predicted using data from only 1-axis of an accelerometer and 1-axis of a gyroscope without the use a magnetometer. Use of this minimized configuration allows determination of the angle of tilt of a person's body with respect to the vertical direction of gravity. Although drift in gyroscope signal are typical, it will likely be minimal in terms of predicting intentions of standing which happens within seconds rather than minutes or hours.

In still another exemplary configuration, physical motion sensor(s) 60 comprise a 9-axis inertial motion unit (IMU). Data collected by the IMU is from a 3-axis accelerometer to detect acceleration (e.g., g-forces) in the x-, y-, and z-directions, a 3-axis gyroscope to detect rotation of a person's body (e.g. roll, pitch, and yaw about the three axes), and a 3-axis magnetometer that measures the Earth's magnetic field and acts like a compass to correct signal drift of the gyroscope signal minimizing error in absolute direction of the individual can also be used. Typical acceleration (e.g., g-force) of a person standing is within +/−2 g range. In the exemplary embodiment, the gyroscope is at a minimum able to detect body rotation of 250 degrees per second, and the magnetometer is be able to detect changes in the magnetic field of 1300 μT. In some implementations, the data from all of the sensors in the IMU needs to be filtered for noise, errors, and drift. This step, however, is unnecessary in some IMUS (such as the commercially available BOSCH BNO055 IMU), as those units fuse all of the sensor outputs together and output a final fusion result vector suitable for training an artificial intelligence system such as predictive AI system 45.

During operation, biometric sensor unit 25″ is, in one embodiment, located on the center of the chest. The chest is preferable as it is the part of the body that has limited motion during sedentary activities (lying or sitting). However, biometric sensor unit 25″ could, during operation, be placed anywhere on the individual, such as the ear, shoulders, wrist, leg, foot, garments such as socks, or on the medical wrist bands.

FIG. 24 is a schematic diagram of an ear mounted biofeedback device 120 according to one particular non-limiting exemplary embodiment of the disclosed concept. As seen in FIG. 23, ear mounted biofeedback device 120 includes an inner ear portion 125 that is connected to a behind the ear housing portion 130 by way of a connecting portion 135. Housing portion 130 houses therein a biofeedback unit (BFU) 137 that is similar in structure and function to biofeedback bedside monitor 95 described herein. BFU 137 includes an audio speaker (located in inner ear portion 125), a controller and a short range and/or long range wireless communication module as described elsewhere herein. Ear mounted biofeedback device 120 may thus be used in any of the embodiments described herein (e.g., any of systems 20, 20′ or 20″) in order to predict the likelihood of experiencing symptoms related to abnormal blood pressure and/or heart rate and/or intention to stand as described in detail herein. Specifically, ear mounted biofeedback device 120 is structured and configured to provide the wearer thereof with real-time cues (e.g., audio cues) to avoid experiencing symptoms related to abnormal blood pressure and/or heart rate based on predictions made by predictive AI system 45 as described herein.

FIG. 25 is a schematic diagram of a necklace biofeedback device 140 according to another particular non-limiting exemplary embodiment of the disclosed concept. As seen in FIG. 25, necklace biofeedback device 140 includes a pendant housing portion 145 that is connected to a chain 150. Pendant housing portion 145 houses therein a BFU 137 as described herein. Necklace biofeedback device 140 may thus be used in any of the embodiments described herein (e.g., any of systems 20, 20′ or 20″) in order to predict the likelihood of experiencing symptoms related to abnormal blood pressure and/or heart rate and/or intention to stand as described in detail herein. Specifically, necklace biofeedback device 140 is structured and configured to provide the wearer thereof with real-time cues (e.g., audio cues) to avoid experiencing symptoms related to abnormal blood pressure and/or heart rate based on predictions made by predictive AI system 45 as described herein. Necklace biofeedback device 140 may, alternatively, take the form of other jewelry such as, without limitation, a brooch, a bracelet, or ear-rings.

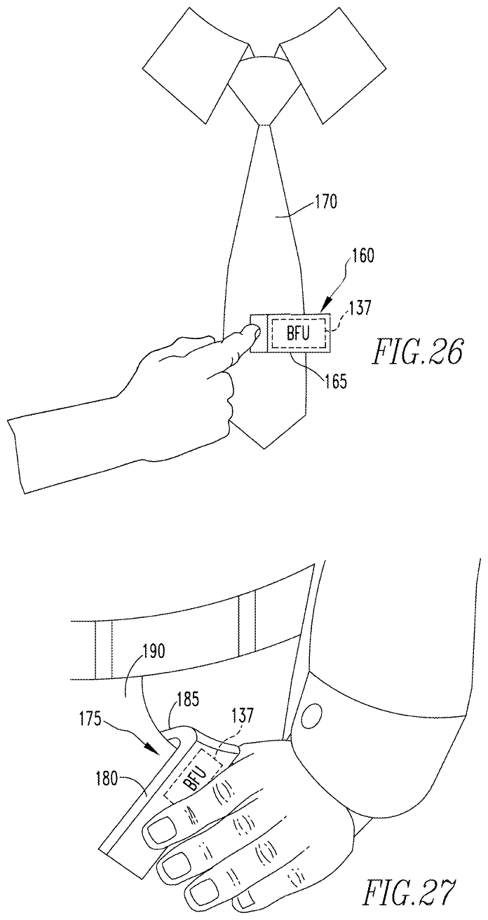

FIG. 26 is a schematic diagram of a tie clip biofeedback device 160 according to another particular non-limiting exemplary embodiment of the disclosed concept. As seen in FIG. 26, tie clip biofeedback device 160 includes a front housing portion 165 that is connected in a biased manner to a rear portion so as to enable tie clip biofeedback device 160 to be readily connected to a tie 170 of a wearer. Front housing portion 165 houses therein a BFU 137 as described herein. Tie clip biofeedback device 160 may thus be used in any of the embodiments described herein (e.g., any of systems 20, 20′ or 20″) in order to predict the likelihood of experiencing symptoms related to abnormal blood pressure and/or heart rate and/or intention to stand as described in detail herein. Specifically, tie clip biofeedback device 160 is structured and configured to provide the wearer thereof with real-time cues (e.g., audio cues) to avoid experiencing symptoms related to abnormal blood pressure and/or heart rate based on predictions made by predictive AI system 45 as described herein.

FIG. 27 is a schematic diagram of a pocket clip biofeedback device 175 according to another particular non-limiting exemplary embodiment of the disclosed concept. As seen in FIG. 27, pocket clip biofeedback device 175 includes a front housing portion 180 that is connected in a biased manner to a rear portion 185 so as to enable pocket clip biofeedback device 175 to be readily connected to a pocket 190 of a wearer as shown. Front housing portion 180 houses therein a BFU 137 as described herein. Pocket clip biofeedback device 175 may thus be used in any of the embodiments described herein (e.g., any of systems 20, 20′ or 20″) in order to predict the likelihood of experiencing symptoms related to abnormal blood pressure and/or heart rate and/or intention to stand as described in detail herein. Specifically, pocket clip biofeedback device 175 is structured and configured provide the wearer thereof with real-time cues (e.g., audio cues) to avoid experiencing symptoms related to abnormal blood pressure and/or heart rate based on predictions made by predictive AI system 45 as described herein. Pocket clip biofeedback device 175 may also be clipped at other locations, such as, without limitation, on the belt or socks of the wearer.

Any of the biofeedback devices shown in FIGS. 24-27 may contain a solid-state audio power amplifier (e.g., a digital speaker), such as Texas Instrument's LM4818, that is capable of providing audible instructions that the person can use to mitigate abnormal blood pressure and/or heart rate episodes and avoid falling. Such devices may also have the ability to provide haptic feedback to warn a person of their fall risk due to experiencing symptoms related to abnormal blood pressure and/or heart rate using a small coin vibration motor, such as the C0720B from Jinlong Machinery. This particular motor measures 7 mm in diameter and 2.1 mm in thickness, provides 0.4 G, and operates within a voltage range of 2.7 to 3.3 VDC.

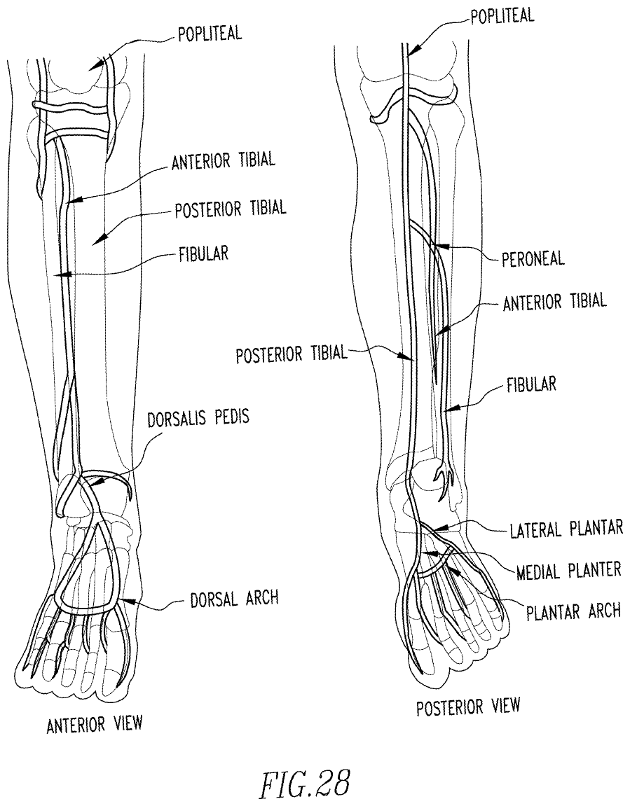

As discussed elsewhere herein, a PPG sensor may be employed to collect heartbeat data for use as described herein. PPG sensors are usually placed in devices that are worn on the wrist, chest, and ears. However, in still a further particular alternative embodiment of the disclosed concept, an alternative location is used for the PPG sensor along the dorsalis pedis and/or posterior tibial arteries available in the lower extremities of the leg as shown in FIG. 28. As shown in the anterior view of FIG. 28, the dorsalis pedis runs along the front of the leg and onto the top of the foot. The posterior tibial artery runs along the back of the leg. Both of these arteries are present in both the left and right legs.

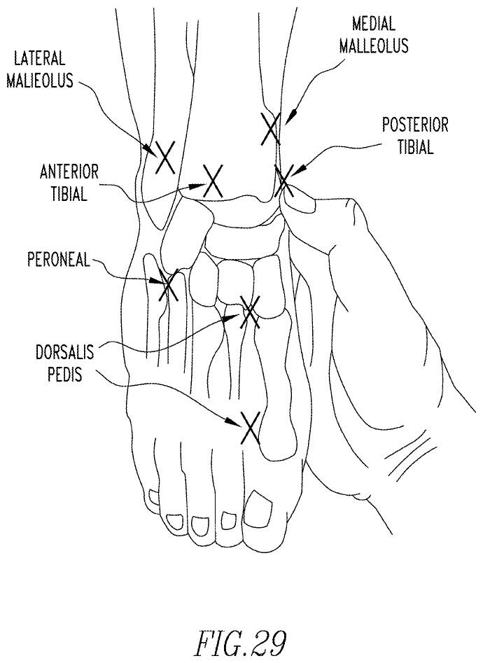

Determination of RR (or NN) intervals using PPG sensors is challenging as it is prone to motion artifacts that deteriorate the training data. Thus, a location, such as the lower leg, where there is minimal movement during sedentary activities (lying, sitting) is desirable. The locations identified by the present inventor to acquire PPG data are along the dorsalis pedis and posterior tibial arteries and they are marked with an “X” in FIG. 29 (i.e., pedal pulse locations). In addition, ambulatory motion of the feet can be predicted using training data from an accelerometer, an accelerometer and gyroscope, or an accelerometer, a gyroscope, and a magnetometer as previously described.

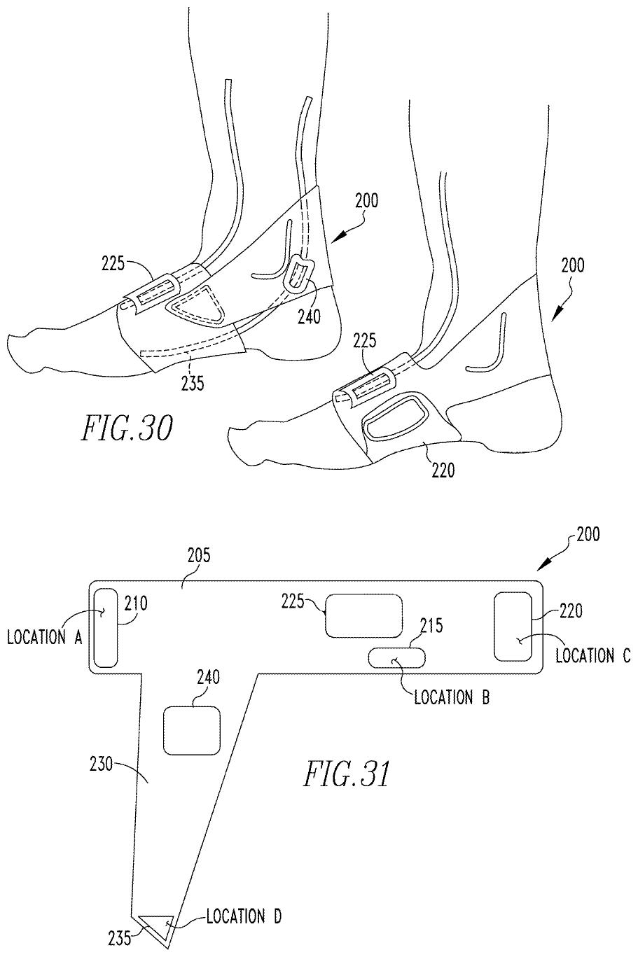

FIGS. 30 and 31 show a pedal pulse sensor device 200 implemented according to this exemplary embodiment of the disclosed concept. As discussed below, pedal pulse sensor device 200 houses therein a number of biometric sensor units 25, 25′ and/or biometric sensor units 25″. Pedal pulse sensor device 200 may thus be used in any of the embodiments described herein (e.g., any of systems 20, 20′ or 20″) in order to predict the likelihood of experiencing symptoms related to abnormal blood pressure and/or heart rate and/or intention to stand as described in detail herein. As seen in FIGS. 30 and 31, in the non-limiting illustrated embodiment, pedal pulse sensor device 200 is a single piece wrap made of a fabric material, such as a cotton/spandex blend. It is equipped with hook and loop (e.g., Velcro™) fastening systems for securing the wrap onto and around each foot. In addition, pedal pulse sensor device 200 includes one or more cavities for each of one or more of the “X” locations shown in FIG. 29 in which a biometric sensor unit 25, 25′, 25″ may reside.

More specifically, as seen in FIGS. 30 and 31, pedal pulse sensor device 200 of the illustrated exemplary embodiment includes a first portion 205 that extends laterally and that includes a hook portion 210 at an end Location A, a hook portion 215 at an intermediate Location B, and a loop portion 220 at an end Location C. A cavity 225 (e.g., a pocket) is provided in the first portion 205 at Location B. Cavity 225 is structured to house a biometric sensor unit 25, 25′, 25″. Pedal pulse sensor device 200 also includes a second portion 230 that extends transversely from first portion 205 proximate to Location A. Second portion 230 includes a loop portion 235 at an end Location D thereof, and a cavity 240 (e.g., a pocket) proximate to the point where second portion 230 connects to first portion 205. Cavity 240 is structured to house a biometric sensor unit 25, 25′, 25″.

In use, pedal pulse sensor device 200 will be wrapped around the foot. Once wrapped around the foot, Location A will meet Location C and Location D will meet Location B (with the hook and loop fasteners as described providing the means for securing to the foot as shown). In the exemplary embodiment, pedal pulse sensor device 200 will be offered in different sizes (e.g., small, medium, large, and extra-large).

In one particular embodiment, pedal pulse sensor device 200 is structured and configured with the capability to detect when an individual's foot has transitioned from the bed and has made a descent towards the floor. To do so, one of the physical motion sensors in biometric sensor unit 25″ held within pedal pulse sensor device 200 is a micro altimeter pressure sensor, such as Servoflo Corporation MS5611-01BA, capable of measuring change in altitude as small as 10 cm. Alternatively, a similar device may be integrated into a sock or worn on the sock either as a clip, snap, or strap, or placed into a cavity sewn into the sock. Accelerometers, gyroscopes, and magnetometers could also be integrated into the system as described herein, with data from these sensors used training the artificial intelligence system to predict when a person has intentions of leaving their bed.

In yet another particular embodiment, a system and method are provided, by way of modification to any of systems 20, 20′ or 20″, for predicting the risk of experiencing symptoms related to abnormal blood pressure and/or heart rate. In this embodiment, subject heart rate variability (HRV) data representing a number of HRV parameters is received, wherein the subject HRV data is generated based on heartbeat data obtained from an individual wearing a heart parameter sensor (such as biometric sensor units 25 and/or biometric sensor units 25′), but only while the individual is in a lying or sitting position prior to standing up. Temporal data changes in or indicated by the received subject HRV data are then analyzed (e.g., by predictive AI system 45) to determine therefrom whether the individual is at risk of experiencing symptoms related to abnormal blood pressure and/or heart rate. If it is determined that the individual is at risk of experiencing symptoms related to abnormal blood pressure and/or heart rate, an output signal is generated that is indicative of the risk level. In this embodiment, the determination as to whether the heartbeat data is only from a period where the individual is in a lying or sitting position prior to standing up is based on motion data collected by a number of physical motion sensors (e.g., sensor(s) 60) worn by the individual.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1-20. (canceled)

21. A method of outputting a warning signal when a subject is at risk of experiencing symptoms related to abnormal blood pressure or heart rate, the method comprising:
obtaining, by a heart parameter sensor worn by a subject, heartbeat data comprising interbeat intervals measured at a frequency of at least 100 Hz;
receiving, by a motion sensor worn by the subject, physical motion data sufficient to distinguish between a resting state and a vertical rising state;
transmitting the heartbeat data and the physical motion data from a wireless transmitter coupled to the sensors to a local computer, wherein the local computer is operative to communicate with the wireless transmitter directly or via a local area network;
identifying, by the local computer, a resting data segment of the heartbeat data by analyzing the physical motion data to isolate data representative of when the subject is in a lying or sitting position;
generating a temporal sequence of heart rate variability (HRV) parameters from the resting data segment;
inputting the temporal sequence of HRV parameters into an artificial intelligence system implemented locally on the local computer, the artificial intelligence system comprising a machine learning model previously trained to identify autonomic nervous system compromise;
determining, by the locally implemented artificial intelligence system, whether the subject is at risk of experiencing an abnormal blood pressure episode based on temporal data changes in the HRV parameters; and
outputting the warning signal from the local computer when the subject is determined to be at risk, wherein the warning signal is output prior to the subject transitioning from the resting state to a standing position.

22. The method of claim 21, wherein the local computer is a smartphone.

23. The method of claim 21, wherein the local computer is operative to communicate with the wireless transmitter directly via a short-range wireless protocol comprising at least one of Bluetooth, ZigBee, and WiFi.

24. The method of claim 21, wherein the physical motion data comprises three-axis acceleration and three-axis rotation.

25. The method of claim 24, further comprising separating the physical motion data into an AC component to detect an intention to stand and a DC component to classify a resting posture.

26. The method of claim 21, wherein the temporal data changes are analyzed within a window of 2 seconds to 5 minutes prior to the subject transitioning to the vertical rising state.

27. The method of claim 21, wherein the HRV parameters include at least one of a Root Mean Square of Successive Differences (RMSSD) and a Power Spectral Density (PSD) in a high-frequency (HF) range of 0.15 to 0.4 Hz.

28. The method of claim 21, further comprising receiving altitude data from a micro altimeter pressure sensor, and wherein identifying the resting data segment further comprises detecting a change in altitude of at least 10 cm.

29. A system for predicting a fall risk related to abnormal blood pressure or heart rate, the system comprising:
a wearable biometric sensor unit comprising a heart parameter sensor structured to generate heartbeat data, a physical motion sensor structured to generate body position data, and a wireless transmitter;
a local computer system comprising a controller and a memory, the local computer system operative to communicate with the wireless transmitter directly or via a local area network;
the local computer system configured to implement an artificial intelligence system previously trained using training heart rate variability (HRV) data and concurrent continuous blood pressure data obtained from a plurality of test subjects;
wherein the local computer system is configured to:
receive the heartbeat data and the body position data from the wearable biometric sensor unit;
sync the heartbeat data with the body position data to identify a portion of the heartbeat data representative of a resting period;
analyze temporal data changes in a plurality of HRV parameters derived from the identified portion of the heartbeat data in the locally implemented artificial intelligence system to determine a risk of the subject experiencing symptoms related to abnormal blood pressure or heart rate; and
trigger a biofeedback alert when the determined risk exceeds a threshold.

30. The system of claim 29, wherein the local computer system is a smartphone.

31. The system of claim 29, wherein the wearable biometric sensor unit is structured to be worn on a chest of the subject.

32. The system of claim 29, wherein the wearable biometric sensor unit is a pedal pulse sensor device comprising a fabric wrap structured to secure around a foot of the subject, the fabric wrap including a cavity positioned to align the heart parameter sensor with a dorsalis pedis artery or a posterior tibial artery.

33. The system of claim 29, wherein the biofeedback alert comprises an actionable cue provided to the subject via the local computer system, the actionable cue comprising an instruction to perform a physical maneuver to mitigate blood pooling.

34. The system of claim 33, wherein the physical maneuver comprises flexing ankles back and forth.

35. The system of claim 29, wherein the biofeedback alert comprises a visual cue displayed on a monitor, the visual cue selected from the group consisting of a “WAIT” symbol and an “OK” symbol.

36. The system of claim 29, wherein the plurality of HRV parameters comprise a Poincaré plot SD1 value representing short-term variation and an SD2 value representing long-term variation.

37. The system of claim 29, wherein the artificial intelligence system comprises a random forest model implementing a collection of decision tree classifiers.

38. A method of training an artificial intelligence system to predict a risk of falling due to symptoms related to abnormal blood pressure, the method comprising:
receiving, at a processor, a training dataset comprising concurrent time-series heartbeat data and continuous arterial blood pressure data obtained from a plurality of test subjects during a plurality of orthostatic stand tests;
extracting, by the processor, a plurality of heart rate variability (HRV) parameters from the heartbeat data;
classifying segments of the extracted HRV parameters with a FALLRISK label by identifying sustained systolic blood pressure drops occurring after a test subject transitions from a resting position to a standing position in the training dataset;
training a recurrent neural network to recognize a signature of autonomic nervous system failure by mapping temporal changes in the classified HRV parameters from a resting period to the labeled FALLRISK segments; and
configuring the trained recurrent neural network to be implemented on a local smartphone to output a real-time prediction of a compromised autonomic state in a subject wearing a heart rate sensor in the absence of concurrent blood pressure measurement.

39. The method of claim 38, wherein the FALLRISK label is applied when the systolic blood pressure drop is at least 20 mmHg.

40. The method of claim 38, further comprising collecting individual-specific heartbeat data and blood pressure data from a specific subject during a subsequent training phase to establish customized risk criteria for the specific subject.
